(19) United States
(12) Patent Application Publication
(10) Pub. No.: US 2017/0000378 A1
(43) Pub. Date: Jan. 5, 2017

(54) MEDICAL IMAGING APPARATUS WITH ILLUMINATION UNIT

(71) Applicant: Siemens Healthcare GmbH, Erlangen (DE)

(72) Inventors: Thomas Kundner, Buckenhof (DE); Markus Petsch, Erlangen (DE); Peter Rupp, Bamberg (DE)

(73) Assignee: Siemens Healthcare GmbH, Erlangen (DE)

(21) Appl. No.: 15/193,709

(22) Filed: Jun. 27, 2016

(30) Foreign Application Priority Data
Jun. 30, 2015 (DE) 102015212204.2

(51) Int. Cl. A61B 5/055; F21V 8/00; G01R 33/54; G01R 33/30; G01R 33/385; F21V 33/00; A61B 5/00

(52) U.S. Cl. CPC A61B 5/055; F21V 33/0068; G02B 6/001; A61B 5/704; G01R 33/307; G01R 33/385; G01R 33/543; G01R 33/546; F21Y 2115/10

(57) ABSTRACT

A medical imaging apparatus, in particular a magnetic resonance apparatus, has a data acquisition scanner, a patient receiving region, which is surrounded at least partially by the data acquisition scanner, and an illumination unit. The illumination unit has a light-generating unit and a light-emitting unit, and the light-generating unit is situated outside the patient receiving region.

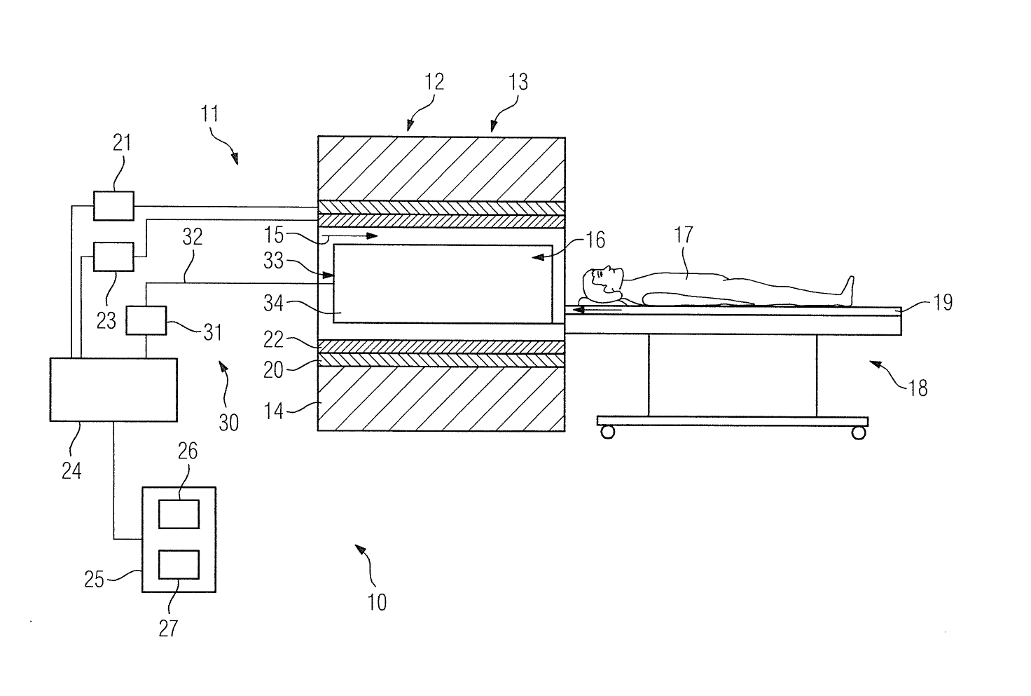

### BACKGROUND OF THE INVENTION

Field of the Invention

The present invention relates to a medical imaging apparatus with a data acquisition scanner, a patient receiving region that is surrounded at least partially by the data acquisition scanner, and that has an illumination unit.

Description of the Prior Art

Medical imaging apparatuses, in particular magnetic resonance apparatuses, have an illumination unit designed to illuminate the patient receiving region. In conventional medical imaging apparatuses, in particular magnetic resonance apparatuses, light-emitting diodes are installed inside the patient receiving region to illuminate the patient receiving region.

### SUMMARY OF THE INVENTION

An object of the present invention is to provide a medical imaging apparatus with which it is possible to prevent interaction between an illumination unit and a data acquisition scanner.

The invention is based on a medical imaging apparatus, in particular a magnetic resonance apparatus, with a data acquisition scanner, having a patient receiving region, which is surrounded at least partially by the data acquisition scanner and an illumination unit.

In accordance with the invention, the illumination unit has a light-generating unit and a light-emitting unit, and the light-generating unit is situated outside the patient receiving region.

In this context, a light-emitting unit means a unit that is preferably designed exclusively for the emission of light, in particular light beams. The emission of the light, in particular the light beams, preferably takes place in the patient receiving region of the medical imaging apparatus. The light-emitting unit is not able to create and/or generate light, in particular light beams. To this end, the light-emitting unit preferably has elements and/or components that are designed for the emission of light, in particular light beams, such as, for example, light-conducting fibers, etc. The design of the light-emitting unit does not use electrical and/or electronic parts (i.e., parts that carry an electrical current).

A light-generating unit as used herein means a unit that is preferably exclusively designed to create and/or generate light, in particular light beams. The emission of the generated light, in particular light beams, for example inside the patient receiving region, by the light-generating does not occur and/is not possible. To this end, the light-generating unit preferably has elements and/or components designed to create and/or generate light, in particular light beams.

The invention facilitates the illumination of the patient receiving region during a medical imaging examination, wherein the light-generating unit of the illumination, and the data acquisition scanner of the medical imaging apparatus, do not interact with one another. This advantageously avoids interference during the acquisition of medical image data and the illumination of the patient receiving region due to the arrangement of the light-generating unit, and hence of electrical and/or electronic components of the illumination unit, outside the patient receiving region. Moreover, this can make a larger space available for the patient inside the patient receiving region.

The invention can achieves a patient environment that is free of electrical and/or electronic components of the illumination unit. This has the additional effect that the emission of waste heat from electrical and/or electronic components of the illumination unit to the patient during a medical imaging examination can be advantageously prevented.

The medical imaging apparatus can be formed by any imaging apparatus considered reasonable to those skilled in the art, such as a computed tomography apparatus, a PET apparatus (positron emission tomography apparatus), etc. However, the medical imaging apparatus is particularly advantageously formed by a magnetic resonance apparatus because in this case interaction between the illumination unit and the data acquisition scanner, in particular a magnet unit with a radio-frequency antenna unit, has particularly severe impacts on imaging.

In an embodiment, the light-emitting unit is a luminous mesh. Such a luminous mesh means a mesh embodying light-conducting elements, for example light-conducting fibers, wherein the light-conducting elements are preferably interwoven in a fabric mesh. This embodiment of the invention facilitates a particularly flexible light-emitting unit, which can be arranged on any surface and/or surface contours. In this case, the light-emitting unit can be adapted to a size and/or shape of further units, in particular the enclosure of the patient receiving region. This can also advantageously provides a magnetic-resonance compatible embodiment of the light-emitting unit.

In a further embodiment of the invention, the light mesh has at least one side-light fiber.

The light mesh preferably has multiple side-light fibers that are woven into the luminous mesh. In this context, a side-light fiber means a light fiber and/or a light conductor with an emission surface for the emission of light, in particular light beams, on a side surface extending in the direction of a longitudinal extension of the side-light fiber. This achieves uniform light emission by the luminous mesh, and a uniform color rendition of the light beams emitted. Moreover, the side-light-conducting fibers inside the luminous mesh advantageously enable different luminous effects, for example differently illuminated sub-areas and/or different colors, in particular different wavelengths, of the emitted light beams to be achieved in a simple way.

In an embodiment, the light-emitting unit is situated inside the patient receiving region, thus advantageously enabling a structurally simple arrangement of the light-emitting unit to illuminate the patient receiving region. In principle, however, a different arrangement of the light-emitting unit, such as, on a patient support table and/or on a housing of the data acquisition scanner and/or further components of the medical imaging apparatus is conceivable.

In a further embodiment of the invention, the light-generating unit has at least one light-emitting diode (LED, light-emitting diode). The light-generating unit preferably has multiple of light-emitting diodes arranged, for example, inside an LED driver, wherein the different light-emitting diodes emit light, in particular light beams, in different colors of light and/or wavelengths, in particular in the primary fundamental colors red, green and blue. This embodiment of the invention facilitates a particularly simple and inexpensive light-generating unit.

In a further embodiment of the invention, the light-generating unit is outside the data acquisition scanner thus, particularly in the case of a magnetic resonance apparatus, enabling unwanted interference between the light-generating unit and the magnet unit to be reduced and/or prevented.

In an alternative embodiment of the invention, the illumination unit has a light transmission unit. This enables an advantageous separation between and/or division of the light-generating unit and the light-emitting unit. In this case, the light transmission unit can be light conductors for the transmission of light, in particular light beams, from the light-generating unit to the light-emitting unit.

Alternatively or additionally, the light transmission unit can be formed in one piece with the light-emitting unit, thus enabling savings on additional parts and/or installation space. In addition, this enables the illumination unit to be embodied particularly inexpensively.

### BRIEF DESCRIPTION OF THE DRAWINGS

The FIGURE is a schematic illustration of a medical imaging apparatus according to the invention with an illumination unit.

### DESCRIPTION OF THE PREFERRED EMBODIMENTS

The figure schematically depicts a medical imaging apparatus 10. In the exemplary embodiment, the medical imaging apparatus 10 is a magnetic resonance apparatus, and the present invention is explained as an example with reference to the magnetic resonance apparatus. However, the present invention is not restricted to an embodiment of the medical imaging apparatus 10 as a magnetic resonance apparatus, and other embodiments of the medical imaging apparatus 10 are conceivable.

The magnetic resonance apparatus has a data acquisition scanner 13 of the medical imaging apparatus 10. The data acquisition scanner has a superconducting basic field magnet 14 for generating a strong and constant basic magnetic field 15. The data acquisition scanner has a patient receiving region 16 for receiving a patient 17. In the exemplary embodiment, the patient receiving region 16 has a cylindrical shape and is enclosed in a circumferential direction in a cylindrical shape by the scanner 13. However, in principle, a different embodiment of the patient receiving region 16 is conceivable. The patient 17 can be moved into the patient receiving region 16 by a patient support 18 of the medical imaging apparatus 10. To this end, the patient support 18 has a patient table 19 that is movable inside the patient receiving region 16.

The scanner 13 further has a gradient coil unit 20 for generating magnetic field gradients, which are used for spatial encoding during imaging. The gradient coil unit 20 is controlled by a gradient control processor 21. The scanner 13 further has a radio-frequency antenna unit 22 for the excitation of nuclear spins in the patient 17 so as to cause the spins to deviate from the polarization that is established in the basic magnetic field 15 generated by the basic field magnet 14. The radio-frequency antenna unit 22 is controlled by a radio-frequency antenna control processor 23 and emits radio-frequency magnetic resonance sequences into an examination chamber substantially formed by the patient receiving region 16.

To control the basic field magnet 14, the gradient control processor 21 and the radio-frequency antenna control processor 23, the medical imaging apparatus 10 has a system control computer 24. The system control computer 24 controls the magnetic resonance apparatus centrally, such as for the performance of a predetermined imaging gradient echo sequence. The system control computer 24 also has an evaluation processor (not shown), for the evaluation of medical image data acquired during the magnetic resonance examination. The medical imaging apparatus 10 also has a user interface 25 connected to the system control computer 24. Control information such as imaging parameters, and reconstructed magnetic resonance images, can be displayed on a display monitor 26, for example on at least one monitor, of the user interface 25 for a medicinal operator. The user interface 25 also has an input unit 27 via which information and/or parameters can be entered by the medical operator during a measuring process.

To illuminate the patient receiving region 16, the data acquisition scanner 13 has an illumination unit 30. In the exemplary embodiment, the illumination unit 30 has a light-generating unit 31, a light transmission unit 32, and a light-emitting unit 33.

The light-generating unit 31 has at least one light-emitting diode. In the exemplary embodiment, the light-generating unit 31 has multiple light-emitting diodes situated inside an LED driver of the light-generating unit 31. In this case, the light-emitting diodes are designed such that light, in particular light beams, in the three fundamental colors red, green and blue can be generated and/or created, and accordingly further colors can be generated by mixing the fundamental colors.

To avoid unwanted interaction between the light-generating unit 31 and the scanner 13, the light-generating unit 31 is situated outside the patient receiving region 16 as well as outside the scanner 13. The light-generating unit 31 is controlled by the system control computer 24.

The light transmission unit 32 is designed to transmit the light generated or created by the light-generating unit 31, in particular light beams generated and/or created thereby, to the light-emitting unit 33. To this end, the light transmission unit 32 comprises light conductors. The light conductors can in addition are be embodied in one piece with light conductors and/or light fibers of the light-emitting unit 33, wherein the light conductors and/or light fibers are bundled inside the light transmission unit 32 and hence arranged in a space-saving manner.

For the emission of light, in particular light beams, the light-emitting unit 33 comprises a luminous mesh 34 composed of light fibers, in particular side-light fibers. The side-light fibers are preferably woven into a fabric mesh and emit the light, in particular the light beams, on an emitting side surface. The emitting side surface preferably extends in the direction of a longitudinal extension, preferably the entire longitudinal extension of the side-light fiber which emit the light, in particular the light beams.

In the exemplary embodiment, the light-emitting unit 33 is arranged inside the patient receiving region 16 so that an as uniform as possible illumination of the patient receiving region 16 can be ensured.

In an alternative embodiment of the invention, the arrangement of the light-emitting unit 33 can also be arranged on the patient support 18, in particular the patient table 19 of the patient support 18. In addition, an arrangement of the light-emitting unit 33 on a housing of the scanner 13 and/or on further units of the magnetic resonance apparatus is also conceivable at any time.

Although modifications and changes may be suggested by those skilled in the art, it is the intention of the inventors to embody within the patent warranted hereon all changes and modifications as reasonably and properly come within the scope of their contribution to the art.

We claim as our invention:

1. A medical imaging apparatus comprising:
a medical data acquisition scanner having a patient-receiving region therein that is at least partially surrounded by said medical data acquisition scanner;
an illumination unit; and
said illumination unit comprising a light-generating unit and a light-emitting unit in optical communication with said light-generating unit, said light-generating unit being situated outside of said patient receiving region.

2. A medical imaging apparatus as claimed in claim 1 wherein said light-emitting unit comprises a luminous mesh.

3. A medical imaging apparatus as claimed in claim 2 wherein said luminous mesh comprises at least one side-light fiber.

4. A medical imaging apparatus as claimed in claim 1 wherein said light-emitting unit is situated inside said patient receiving region.

5. A medical imaging apparatus as claimed in claim 1 wherein said light-generating unit comprises at least one light-emitting diode.

6. A medical imaging apparatus as claimed in claim 1 wherein said light-generating unit is situated outside of said medical data acquisition scanner.

7. A medical imaging apparatus as claimed in claim 1 wherein said illumination unit comprises a light transmission unit between said light-emitting unit and said light-generating unit.

8. A medical imaging apparatus as claimed in claim 7 wherein said light transmission unit is formed as one piece with said light-emitting unit.

9. A medical imaging apparatus as claimed in claim 1 wherein said medical data acquisition scanner is a magnetic resonance data acquisition scanner.
